(19) United States
(12) Patent Application Publication
(10) Pub. No.: US 2021/0097844 A1
(43) Pub. Date: Apr. 1, 2021

(54) Accelerometer-Based Ergonomic Counseling by a Hearing Device

(71) Applicant: SONOVA AG, Staefa (CH)

(72) Inventor: Nadim El Guindi, Zürich (CH)

(21) Appl. No.: 16/585,215

(22) Filed: Sep. 27, 2019

(51) Int. Cl. G08B 21/24; H04S 7/00; G10L 25/51; H04R 25/00

(52) U.S. Cl. CPC G08B 21/24; H04S 7/303; H04R 2225/67; H04R 25/552; H04S 2420/01; G10L 25/51

(57) ABSTRACT

An exemplary hearing device is configured to be worn by a user and includes an accelerometer data configured to output accelerometer data associated with the hearing device. The hearing device is configured to monitor the accelerometer data, determine, based on the monitored accelerometer data, that a head of the user is in an ergonomically incorrect position, and perform, based on the determination that the head of the user is in the ergonomically incorrect position, a remedial action configured to prompt the user to move the head to an ergonomically correct position.

### BACKGROUND INFORMATION

People are increasingly spending time in ergonomically incorrect positions that may lead to injury and/or pain. For example, mobile device users often hold their head tilted and/or rotated with respect to a longitudinal axis of their body while using mobile devices. Extended time spent in these positions may lead to conditions such as “text neck” (or “text neck syndrome”) and/or “forward head posture.” Such conditions may also be caused by ergonomically incorrect positions held for prolonged periods of time while using a computer, sleeping incorrectly, or for other reasons. It may be desirable to be able to detect ergonomically incorrect positions and provide cues or other ergonomic counseling to prevent such prolonged time spent in ergonomically incorrect positions.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an exemplary configuration of a hearing device according to principles described herein.

FIG. 2 illustrates an exemplary configuration in which a hearing device is configured to provide ergonomic counseling according to principles described herein.

FIGS. 3A and 3B illustrate exemplary ergonomically correct and incorrect positions according to principles described herein.

FIG. 4 illustrates an exemplary configuration of a binaural hearing device according to principles described herein.

FIG. 5 illustrates an exemplary configuration of a hearing device in communication with a mobile device according to principles described herein.

FIG. 6 illustrates an exemplary method according to principles described herein.

### DETAILED DESCRIPTION

Accelerometer-based ergonomic counseling by a hearing device is described herein. For example, a hearing device configured to be worn by a user may include an accelerometer configured to output accelerometer data associated with the hearing device and a processor communicatively coupled to the accelerometer. The processor may be configured to monitor the accelerometer data, determine, based on the monitored accelerometer data, that a head of the user is in an ergonomically incorrect position, and perform, based on the determination that the head of the user is in the ergonomically incorrect position, a remedial action configured to prompt the user to move the head to an ergonomically correct position.

For example, the processor may be configured to determine that the head of the user is in the ergonomically incorrect position by determining that the head is tilted and/or rotated with respect to a longitudinal axis of a body of the user. The processor may be configured to perform the remedial action by providing an audible notification via the hearing device and/or by providing a notification to a mobile device.

The systems, hearing devices, and methods described herein may advantageously provide many benefits to a user of a hearing device. For example, the systems, hearing devices, and methods described herein may allow a hearing device to detect ergonomically incorrect positions of the user. The hearing device may provide cues, prompts, notifications, etc. that may counsel the user to avoid such ergonomically incorrect positions. Such prompts may allow the user to avoid injury and/or pain associated with prolonged holding of ergonomically incorrect positions. These and other benefits of the systems, hearing devices, and methods described will be made apparent herein.

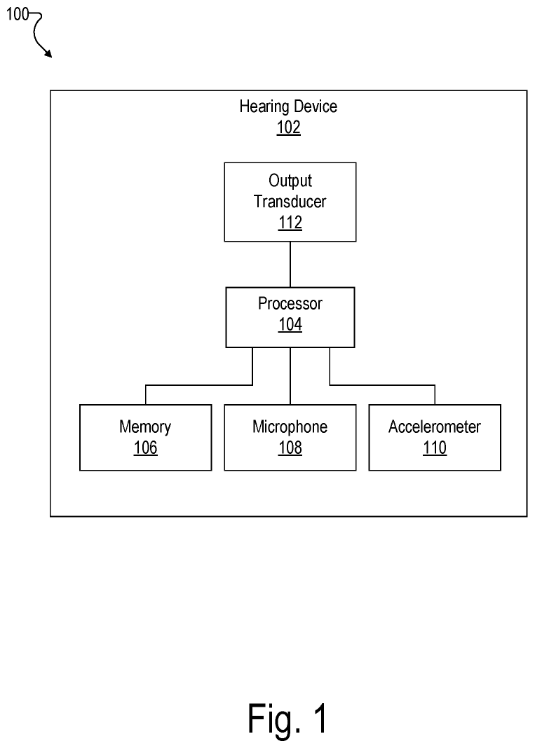

FIG. 1 shows an exemplary configuration of a hearing device 102 for providing ergonomic counseling. Hearing device 102 may be implemented by any type of device configured to provide or enhance hearing to a user. For example, hearing device 102 may be implemented by a hearing aid configured to provide an audible signal (e.g., amplified audio content) to a user, a sound processor included in a cochlear implant system configured to apply electrical stimulation representative of audio content to a user, a sound processor included in a system configured to apply both acoustic and electrical stimulation to a user, or any other suitable hearing prosthesis. As shown, hearing device 102 includes a processor 104 communicatively coupled to a memory 106, a microphone 108, an accelerometer 110, and an output transducer 112. Hearing device 102 may include additional or alternative components as may serve a particular implementation.

Microphone 108 may be implemented by any suitable audio detection device and is configured to detect audio content ambient to a user of hearing device 102. Microphone 108 may be included in or communicatively coupled to hearing device 102 in any suitable manner.

Accelerometer 110 may be implemented by any suitable sensor configured to detect movement (e.g., acceleration) of hearing device 102. While hearing device 102 is being worn by a user, the detected movement of hearing device 102 is representative of movement by the user.

Output transducer 112 may be implemented by any suitable audio output device, such as a loudspeaker of a hearing device or an output electrode of a cochlear implant system.

Memory 106 may be implemented by any suitable type of storage medium and may be configured to maintain (e.g., store) data generated, accessed, or otherwise used by processor 104. For example, memory 106 may maintain accelerometer data, data representative of ergonomically correct and incorrect positions, potential remedial actions, etc.

Processor 104 may be configured to perform various processing operations with respect to providing ergonomic counseling for a user. For example, processor 104 may be configured to monitor and analyze accelerometer data generated by accelerometer 110. Processor 104 may be configured to perform various remedial actions based on the accelerometer data. Example implementations and other operations that may be performed by processor 104 are described in more detail herein. In the description that follows, any references to operations performed by hearing device 102 may be understood to be performed by processor 104 of hearing device 102.

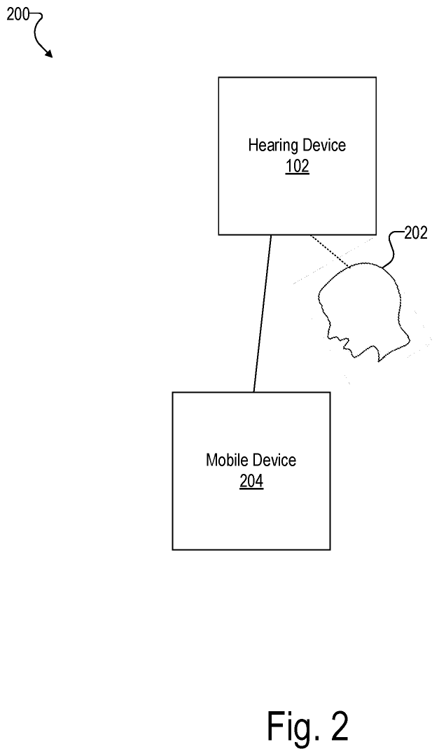

FIG. 2 illustrates an exemplary configuration 200 in which hearing device 102 is configured to provide ergonomic counseling. As shown, hearing device 102 is worn by a user 202 to enable or enhance hearing by user 202. Hearing device 102 is configured to connect to a mobile device 204.

Mobile device 204 may be implemented by any suitable type of mobile computing device that is capable of being communicatively coupled to hearing device 102 and performing any of the operations described herein. For example, mobile device 204 may be implemented by a handheld device (e.g., a mobile phone such as a smartphone, a tablet computer, a mobile gaming device, a personal medical device, a controller for hearing device 102, etc.), a personal computer (e.g., a laptop computer), or a wearable device (e.g., a smartwatch, a fitness tracker, a head-mounted display device, etc.). Mobile device 204 may include any hardware and/or software that enable mobile device 204 to perform one or more of the operations described herein. For example, mobile device 204 may maintain and selectively execute a software application configured to control and/or communicate with hearing device 102.

A head of user 202 may be in an ergonomically incorrect position while using mobile device 204. Exemplary ergonomically incorrect positions are described further herein. Hearing device 102 may detect that the head of user 202 is in the ergonomically incorrect position and perform a remedial action configured to prompt user 202 to move his/her head to an ergonomically correct position. Hearing device 102 may perform the remedial action in any suitable manner.

As an example, hearing device 102 may perform the remedial action by providing an audible notification to user 202 (e.g., via output transducer 112). To illustrate, hearing device 102 may provide an audible message indicating that the head of user 202 is in an ergonomically incorrect position in a certain direction and/or a suggestion that user 202 move his/her head in a certain direction. Additionally or alternatively, hearing device 102 may perform the remedial action by providing a notification to mobile device 204. For example, hearing device 102 may direct mobile device 204 to present (e.g., display and/or audibly play) a notification indicating that user 202 should move his/her head in a certain direction. Additionally or alternatively, hearing device 102 may perform the remedial action by providing a command to mobile device 204 to turn off a screen of mobile device 204 for a period of time. These and other exemplary remedial actions are further described herein.

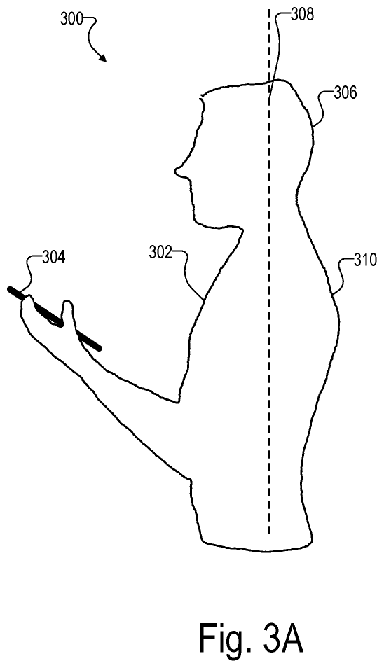

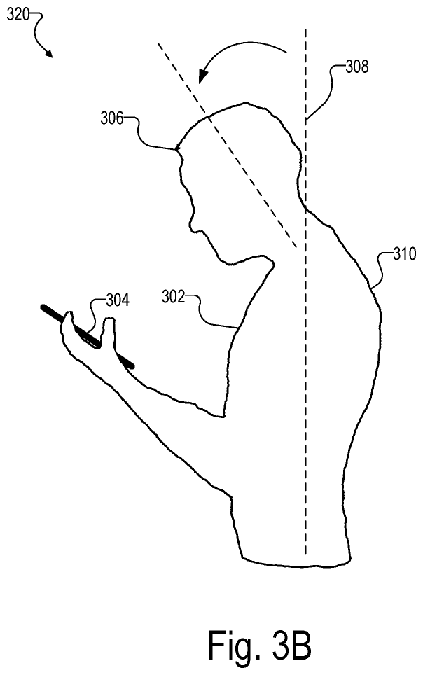

FIGS. 3A and 3B illustrate exemplary ergonomically correct and incorrect positions of a user 302, respectively. FIG. 3A shows a position 300 of user 302 using a mobile device 304. A head 306 of user 302 is upright, in line with a longitudinal axis 308 of a body 310 of user 302. Such a position of head 306 in line with longitudinal axis 308 of body 310 of user 302 may be an example ergonomically correct position. Ergonomically correct positions may be any positions in which body parts are aligned such that strain on the body parts is minimized. As a counterexample, FIG. 3B shows a position 320 of user 302 using mobile device 304. Head 306 of user 302 is bent, such that head 306 of user 302 is tilted with respect to longitudinal axis 308 of body 310 of user 302. Such a position (sometimes referred to as “text neck”) may be an example ergonomically incorrect position. Ergonomically incorrect positions may be any positions in which body parts are not aligned, which may lead to pain and/or injury as the positions are held and/or repeated over time.

While position 320 shows head 306 of user 302 tilted with respect to longitudinal axis 308 in such a manner that head 306 is facing down, another related example ergonomically incorrect position is a “forward head posture” position. In such a position, head 306 may be tilted with respect to longitudinal axis 308 while head 306 still faces forward, resulting in a forward protrusion of head 306. The forward head posture position may often be an ergonomically incorrect position held while using a laptop computer, or a result of other causes such as reading, sleeping in ergonomically incorrect positions, etc. Additionally or alternatively, while position 320 shows head 306 tilted forward with respect to longitudinal axis 308, other ergonomically incorrect positions may include head 306 tilted in any direction with respect to longitudinal axis 308. Additionally or alternatively, some example ergonomically incorrect positions may include a rotation of head 302. While a rotated position of head 302 may allow head 302 to remain aligned with respect to longitudinal axis 308, the rotation may still result in pain and/or injury and thus may be considered as an ergonomically incorrect position.

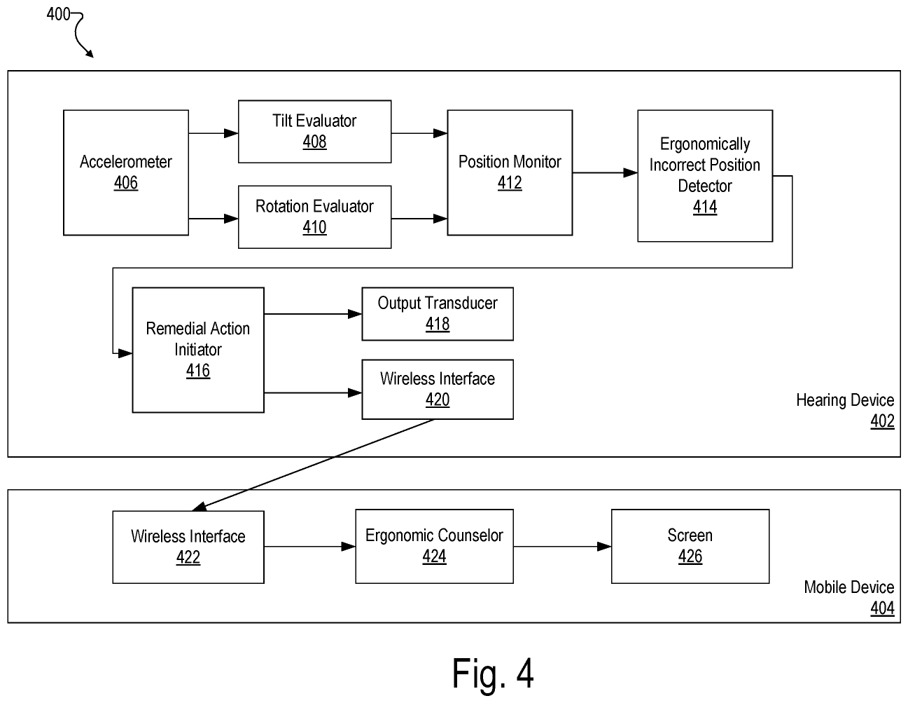

A hearing device may detect, using accelerometer data, positions of head 306 of user 302 and determine whether the positions are ergonomically correct or incorrect. FIG. 4 shows an exemplary configuration 400 of a hearing device 402 communicatively coupled to a mobile device 404. Hearing device 402 may be an implementation of hearing device 102. Mobile device 404 may be an implementation of mobile device 204.

As shown, hearing device 402 includes an accelerometer 406, a tilt evaluator 408, a rotation evaluator 410, a position monitor 412, an ergonomically incorrect position detector 414, a remedial action initiator 416, an output transducer 418, and a wireless interface 420. Each of these components may be implemented by any suitable combination of hardware and/or software.

Accelerometer 406 (e.g., an implementation of accelerometer 110) generates accelerometer data. As described, while a user is wearing hearing device 402, accelerometer 406 may generate accelerometer data representative of movement of the user's head. By monitoring the accelerometer data, hearing device 402 may detect positions of the user's head and determine whether the positions are ergonomically correct or incorrect. Hearing device 402 may detect positions of the user's head using tilt evaluator 408 and rotation evaluator 410.

Tilt evaluator 408 may access (e.g., receive) accelerometer data from accelerometer 406. Tilt evaluator 408 may analyze the accelerometer data to determine whether the user's head is tilted. For example, tilt evaluator 408 may determine that the user's head is tilted if the accelerometer data shows the user's head has moved a threshold amount in various directions and/or on various axes (and/or has not moved in other directions and/or on other axes) that may be indicative of a tilting of the head with respect to a longitudinal axis of a body of the user.

Rotation evaluator 410 may also access (e.g., receive) accelerometer data from accelerometer 406. Rotation evaluator 410 may analyze the accelerometer data to determine whether the user's head is rotated. For example, rotation evaluator 410 may determine that the user's head is rotated if the accelerometer data shows the user's head has moved in a horizontal plane while remaining relatively still in a vertical plane.

In some instances, tilt evaluator 408 and rotation evaluator 410 may receive accelerometer data from another hearing device. For example, hearing device 402 may be one of two hearing devices of a binaural hearing system. In such a binaural hearing system, accelerometer data from both sides of a user's head may be used together to more accurately determine tilt and rotation of the user's head. An example binaural hearing system is described further herein.

Position monitor 412 may access (e.g., receive) information from tilt evaluator 408 and rotation evaluator 410 to determine and monitor a position of the user's head. For example, position monitor 412 may combine results from tilt evaluator 408 and rotation evaluator 410 temporally to determine positions of the user's head at a given time. Position monitor 412 may also store position data to analyze the user's head positions over periods of time. Such monitoring may be relevant as time spent in ergonomically incorrect positions may result in pain and/or injury as the time increases. Consequently, hearing device 402 may provide remedial actions that are based on time information (e.g., a duration of time the user's head has been in an ergonomically incorrect position, a number of times the user's head has been in an ergonomically incorrect position, an aggregated threshold amount of time over a predetermined time period, etc.), as will be described herein.

Ergonomically incorrect position detector 414 may access (e.g., receive) information from position monitor 412 and detect ergonomically incorrect positions. For example, ergonomically incorrect position detector 414 may analyze the positions of the user's head as determined by position monitor 412 to recognize specific ergonomically incorrect positions, such as text neck and/or forward head posture. Alternatively or additionally, ergonomically incorrect position detector 414 may analyze the positions to find positions where the head is a threshold amount of tilt and/or rotation is met, indicating that the head is not in an ergonomically correct position. Ergonomically incorrect position detector 414 may provide an indication to remedial action initiator 416 upon detection of an ergonomically incorrect position. Ergonomically incorrect position detector 414 may also provide time information to remedial action initiator 416. Additionally or alternatively, remedial action initiator 416 may access time information from position monitor 412.

Remedial action initiator 416 may receive an indication from ergonomically incorrect position detector 414 that the user's head is in an ergonomically incorrect position. Based on receiving the indication, remedial action initiator 416 may initiate a remedial action to be performed by hearing device 402.

Remedial actions may include any action configured to prompt the user to move his/her head to an ergonomically correct position. For example, remedial actions may include audible notifications provided by hearing device 402 (e.g., via output transducer 418). Audible notifications may include a sound (e.g., a bell, a ding, a sound effect, etc.) configured by the user to serve as reminder to fix his/her posture (e.g., by moving his/her head to an ergonomically correct position). Additionally or alternatively, audible notifications may include a message directed toward the user to move his/her head to an ergonomically correct position. For instance, the message may tell the user to “sit up,” or “take a break,” or remind the user “your head is tilted down” or provide specific direction to the user such as “rotate your head to the left.”

Additionally or alternatively, audible notifications may be provided from a virtual direction. For example, hearing device 402 may provide a sound (e.g., a bell, a bird chirping, a voice, etc.) to the user that appears to be coming from above the user's head or from directly in front of the user to prompt the user to look up, moving his/her head into an ergonomically correct position. While the sound is provided by hearing device 402, which is worn by the user in a specific location, hearing device 402 may configure the sound to appear as if coming from a specific direction using any suitable techniques. As an example, hearing device 402 may configure the sound using a head-related transfer function (or a pair of head-related transfer functions for a binaural hearing system) to synthesize how the sound would arrive at the ear from a specific point or direction.

Additionally or alternatively, remedial actions may include actions with respect to mobile device 404. Hearing device 402 may communicate with mobile device 404 in any suitable manner. For example, wireless interface 420 on hearing device 402 may communicate with a wireless interface 422 on mobile device 404. Mobile device 404 also includes an ergonomic counselor 424 and a screen 426. Ergonomic counselor 424 may be implemented in any suitable manner. For example, ergonomic counselor 424 may be a software application running on mobile device 404 that may provide notifications and prompts to the user, as well as providing information to hearing device 402. For instance, ergonomic counselor 424 may indicate to hearing device 402 whether screen 426 is on or off. Hearing device 402 (e.g., remedial action initiator 416) may perform remedial actions and/or choose remedial actions to perform based on whether screen 426 is on. If screen 426 is off, remedial action initiator 416 may choose to perform remedial actions via output transducer 418, such as audible notifications. If screen 426 is on, remedial action initiator 416 may perform remedial actions with respect to mobile device 404, especially if position monitor 412 and/or ergonomically incorrect position detector 414 indicate the user is likely in an ergonomically incorrect position due to usage of mobile device 404.

Example remedial actions with respect to mobile device 404 may include notifications shown on screen 426 (e.g., messages similar to the audible messages provided via output transducer, visual cues, etc.). Additionally or alternatively, remedial actions with respect to mobile device 404 may include changing a display on screen 426 (e.g., greying/whiting/blanking out screen 426). Additionally or alternatively, remedial actions with respect to mobile device 404 may include turning off screen 426.

As mentioned, remedial action initiator 416 may initiate remedial actions based on time information. For example, remedial action initiator 416 may wait to initiate a remedial action until a threshold amount of time is met that the user is in an ergonomically incorrect position. Additionally or alternatively, remedial action initiator 416 may initiate the remedial action upon determining that the user's head is in the ergonomically incorrect position for an aggregated threshold amount of time over a predetermined time period. Additionally or alternatively, remedial action initiator 416 may determine different remedial actions based on time information. For example, hearing device 402 may perform a mild remedial action after a first threshold time period, such as an audible prompt. After a second threshold time period, hearing device 402 may perform a stronger remedial action, such as an audible message. After a third threshold time period, hearing device 402 may perform an even stronger remedial action, such as turning off screen 426 of mobile device 404. After an aggregated threshold amount of time is met, hearing device 402 may perform a remedial action as soon as the head is determined to be in the ergonomically incorrect position, or within a threshold amount of time of the determination. While one example has been provided, any other suitable combinations of time information and remedial actions may be used.

While FIG. 4 shows hearing device 402 in communication with mobile device 404, hearing device 402 may provide ergonomic counseling independent of mobile device 404. For example, a user may be in an ergonomically incorrect position while sitting at a desk at work. Hearing device 402 may provide audible notifications as described to prompt the user to move his/her head to an ergonomically correct position.

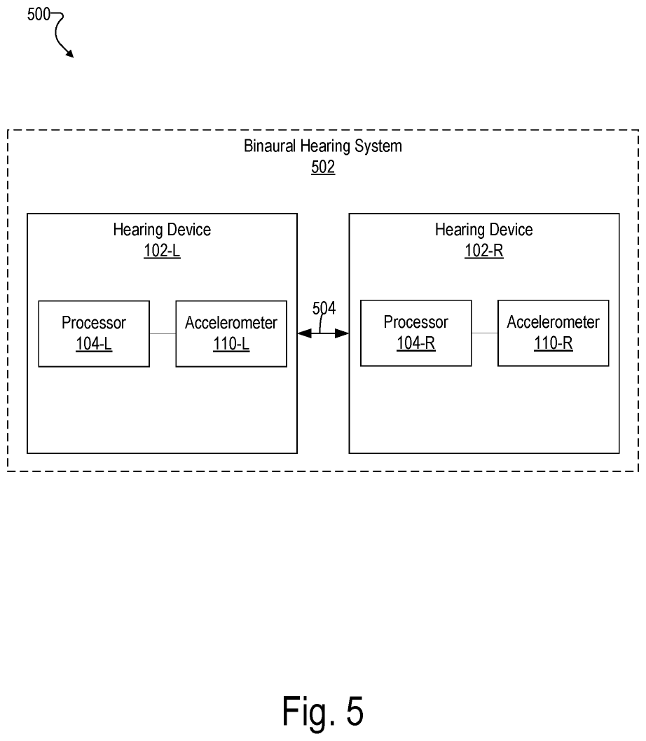

FIG. 5 illustrates an exemplary configuration 500 in which a binaural hearing system 502 is configured to provide ergonomic counseling based on accelerometer data. As shown, binaural hearing system 502 includes a left hearing device 102-L and a right hearing device 102-R (collectively “hearing devices 102”). Each of hearing devices 102 may be implemented as described above in FIG. 1, 2, or 4. Alternatively, binaural hearing system 502 may include asymmetric left and right hearing devices. For example, one or more of the components such as the accelerometer, microphone, etc. may each be included in only one or the other of hearing devices 502. The hearing device with the component may communicate with the other hearing device (as indicated by arrow 504) to provide accelerometer data, head-related transfer function data, audio data, etc.

As shown, hearing devices 102 each include an accelerometer 110 (e.g., accelerometer 110-L or accelerometer 110-R), which may enable binaural hearing system 502 to accurately determine a movement of a user. For example, by analyzing a slope, value, sign, etc. of accelerometer data received from both accelerometer 110-L and 110-R, binaural hearing system 502 may determine which direction a user tilts and/or rotates his/her head. Binaural hearing system 502 may then analyze the accelerometer data to determine ergonomically correct and incorrect positions of the user's head and provide ergonomic counseling accordingly.

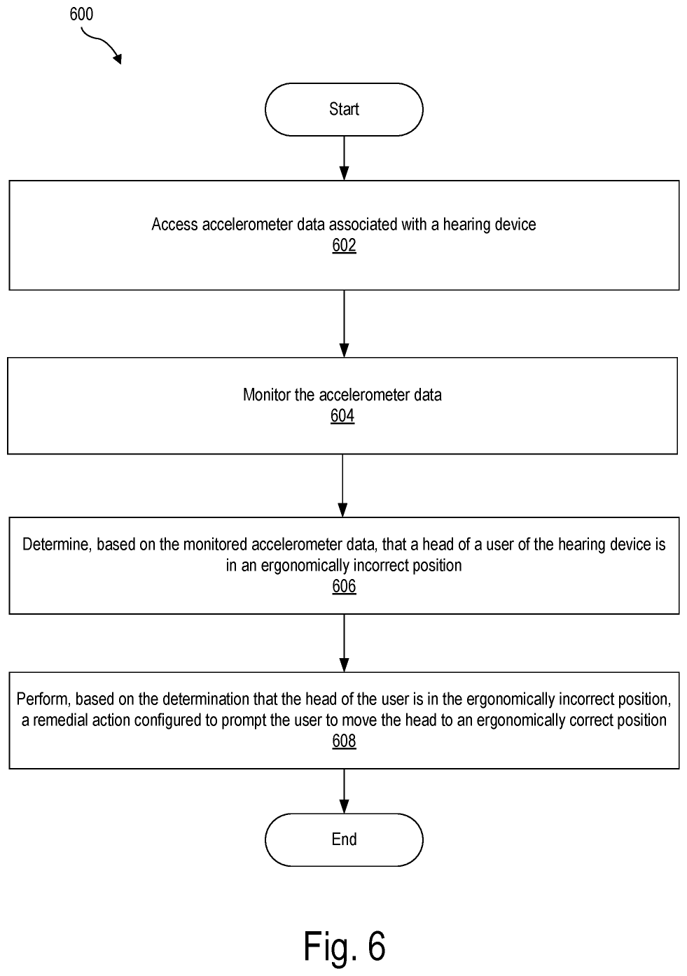

FIG. 6 illustrates an exemplary method 600. One or more of the operations shown in FIG. 6 may be performed by any of the hearing devices described herein. While FIG. 6 illustrates exemplary operations according to one embodiment, other embodiments may omit, add to, reorder, and/or modify any of the operations shown in FIG. 6.

In step 602, a hearing device configured to be worn by a user accesses accelerometer data associated with the hearing device. Step 602 may be performed in any of the ways described herein.

In step 604, the hearing device monitors the accelerometer data. Step 604 may be performed in any of the ways described herein.

In step 606, the hearing device determines, based on the monitored accelerometer data, that a head of the user of the hearing device is in an ergonomically incorrect position. Step 606 may be performed in any of the ways described herein.

In step 608, the hearing device performs, based on the determination that the head of the user is in the ergonomically incorrect position, a remedial action configured to prompt the user to move the head to an ergonomically correct position. Step 608 may be performed in any of the ways described herein.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A hearing device configured to be worn by a user, the hearing device comprising:
an accelerometer configured to output accelerometer data associated with the hearing device;
a processor communicatively coupled to the accelerometer, the processor configured to:
monitor the accelerometer data;
determine, based on the monitored accelerometer data, that a head of the user is in an ergonomically incorrect position; and
perform, based on the determination that the head of the user is in the ergonomically incorrect position, a remedial action configured to prompt the user to move the head to an ergonomically correct position by:
configuring a sound using a head-related transfer function to synthesize the sound to appear to come from a specific direction, and
providing the sound to the user.

2. The hearing device of claim 1, wherein the processor is configured to determine that the head of the user is in the ergonomically incorrect position by determining that the head is tilted with respect to a longitudinal axis of a body of the user.

3. The hearing device of claim 1, wherein the processor is configured to determine that the head of the user is in the ergonomically incorrect position by determining that the head is rotated with respect to a longitudinal axis of a body of the user.

4. The hearing device of claim 1, wherein the sound is an audible notification to move the head to the ergonomically correct position.

5-6. (canceled)

7. The hearing device of claim 1, wherein the processor is further configured to perform the remedial action by providing a notification to a mobile device.

8. The hearing device of claim 1, wherein the processor is further configured to perform the remedial action by providing a command to a mobile device to turn off a screen of the mobile device.

9. The hearing device of claim 1, wherein:
the processor is further configured to determine that a screen of a mobile device associated with the user is turned on while the head of the user is in the ergonomically incorrect position;
the performing of the remedial action is further based on the determination that the screen is turned on while the head of the user is in the ergonomically incorrect position; and
the remedial action is with respect to the mobile device.

10. The hearing device of claim 1, wherein:
the performing the remedial action occurs upon determining that the head is in the ergonomically incorrect position for a threshold duration of time.

11. The hearing device of claim 1, wherein:
the performing the remedial action occurs upon determining that the head is in the ergonomically incorrect position for an aggregated threshold amount of time over a predetermined time period.

12. The hearing device of claim 1, wherein the processor is further configured to:
perform, based on determining that the head is in the ergonomically incorrect position for a threshold amount of time, a second remedial action configured to prompt the user to move the head to the ergonomically correct position.

13. The hearing device of claim 1, further comprising:
a microphone communicatively coupled to the processor and configured to detect an audio signal; and
an output transducer communicatively coupled to the processor and configured to output an audio output signal based on the audio signal.

14. A hearing system comprising:
a first hearing device configured for a first ear of a user, the first hearing device comprising a first accelerometer configured to output first accelerometer data associated with the first hearing device;
a second hearing device configured for a second ear of the user, the second hearing device comprising a second accelerometer configured to output second accelerometer data associated with the second hearing device;
a processor communicatively coupled to the first hearing device and the second hearing device, the processor configured to:
monitor the first accelerometer data and the second accelerometer data;
determine, based on the monitored first and second accelerometer data, that a head of the user is in an ergonomically incorrect position; and
perform, based on the determination that the head of the user is in the ergonomically incorrect position, a remedial action configured to prompt the user to move the head to an ergonomically correct position by:
configuring a sound using a head-related transfer function to synthesize the sound to appear to come from a specific direction, and
providing the sound to the user.

15. The hearing system of claim 14, wherein the processor is configured to determine that the head of the user is in the ergonomically incorrect position by determining that the head is at least one of:
tilted with respect to a longitudinal axis of a body of the user, and
rotated with respect to the longitudinal axis of the body of the user.

16. The hearing system of claim 14, wherein the sound is an audible notification to move the head to the ergonomically correct position.

17. (canceled)

18. The hearing system of claim 14, wherein the processor is further configured to perform the remedial action by providing a notification to a mobile device.

19. The hearing system of claim 14, wherein the processor is further configured to perform the remedial action upon determining that the head is in the ergonomically incorrect position for a threshold duration of time.

20. A method comprising:
accessing, by a hearing device configured to be worn by a user, accelerometer data associated with the hearing device;
monitoring, by the hearing device, the accelerometer data;
determining, by the hearing device, based on the monitored accelerometer data, that a head of the user is in an ergonomically incorrect position; and
performing, by the hearing device, based on the determination that the head of the user is in the ergonomically incorrect position, a remedial action configured to prompt the user to move the head to an ergonomically correct position by:
configuring a sound using a head-related transfer function to synthesize the sound to appear to come from a specific direction, and
providing the sound to the user.

21. The hearing device of claim 1, wherein the specific direction is in front of the user.

22. The hearing device of claim 1, wherein the specific direction is above the head of the user.

23. The hearing system of claim 14, wherein the specific direction is in front of the user or above the head of the user.
